(19) United States
(12) Patent Application Publication
(10) Pub. No.: US 2022/0378518 A1
(43) Pub. Date: Dec. 1, 2022

(54) SURGICAL ROBOT

(71) Applicant: RIVERFIELD INC., Tokyo (JP)

(72) Inventors: Masao KANAZAWA, Tokyo (JP); Yasushi TANAKA, Tokyo (JP)

(73) Assignee: RIVERFIELD INC., Tokyo (JP)

(21) Appl. No.: 17/885,111

(22) Filed: Aug. 10, 2022

Related U.S. Application Data
(63) Continuation of application No. PCT/JP2021/000706, filed on Jan. 12, 2021.

(30) Foreign Application Priority Data
Feb. 12, 2020 (JP) 2020-021632

(51) Int. Cl. A61B 34/20; A61B 34/30; A61B 17/34; A61B 1/00; A61B 46/10

(52) U.S. Cl. CPC A61B 34/20; A61B 34/30; A61B 17/34; A61B 1/00045; A61B 46/10; A61B 2034/301

(57) ABSTRACT

A surgical robot for use in endoscopic surgery includes an arm device that holds a surgical instrument used in endoscopic surgery, a drive device that drives the arm device, a display section, and a display processor that displays, on the display section, a relative positional relationship between a trocar site and a distal end position of the surgical instrument.

### CROSS-REFERENCE TO RELATED APPLICATION

This U.S. application is a continuation of International Application No. PCT/JP2021/000706, filed Jan. 12, 2021, which is based on and claims priority from Japanese Patent Application No. 2020-021632 filed on Feb. 12, 2020 with the Japan Patent Office, and the contents of each of which being herein incorporated by reference herein in their entireties.

### BACKGROUND

The present disclosure relates to a surgical robot for use in endoscopic surgery.

During endoscopic surgery such as laparoscopic surgery, a surgical instrument such as an endoscope and forceps is inserted into a body of the subject through an incision position, that is, a site where a trocar is to be inserted. Thus, it is necessary for the surgical robot to move the surgical instrument so that a portion of the surgical instrument corresponding to the incision site is immovable. However, it can be difficult to determine whether the surgical instrument is moving so that the portion of the surgical instrument corresponding to the incision site is immovable.

### SUMMARY

It is an aspect to provide a surgical robot that allows for confirmation of whether the surgical instrument is moving or not so that the portion of the surgical instrument corresponding to the incision site is immovable.

According to an aspect of one or more embodiments, there is provided a surgical robot comprising an arm device that holds a surgical instrument used in endoscopic surgery; a drive device that drives the arm device; a display section; and a first display processor that displays, on the display section, a relative positional relationship between a site where a trocar is to be inserted and a distal end position of the surgical instrument.

According to an aspect of one or more embodiments, there is provided a surgical robot comprising a robot arm that holds a surgical instrument used in endoscopic surgery; a drive device that drives the robot arm; a display; and a processor or hardware logic that displays, on the display, a relative positional relationship between a trocar site and a distal end position of the surgical instrument.

According to an aspect of one or more embodiments, there is provided a surgical robot comprising a robot arm that holds a surgical instrument used in endoscopic surgery; a drive device that drives the robot arm; a display; and a processor or hardware logic that based on a setting button being depressed, starts an immovable point setting mode that allows free displacement of the robot arm; based on the setting button being depressed again, recognizes a position of a site where a trocar is to be inserted, stores the position as the trocar site, and ends the immovable point setting mode; and displays, on the display, a relative positional relationship between the trocar site and a distal end position of the surgical instrument.

### BRIEF DESCRIPTION OF THE DRAWINGS

The above and/or other aspects will become apparent and more readily appreciated from the following description of various embodiments, taken in conjunction with the accompanying drawings of which:

FIG. 1 is an external view of a surgical robot according to some embodiments;

FIG. 2 is a block diagram of the surgical robot according to some embodiments;

FIGS. 3A to 3C are diagrams showing display examples of a first display processor;

FIGS. 4A and 4B are diagrams showing display examples of a second display processor; and

FIG. 5 is a flowchart showing a control of an immovable point setting mode of the surgical robot according to some embodiments.

### DETAILED DESCRIPTION

Endoscopic surgery such as laparoscopic surgery is performed by the following procedure.

Specifically, an operator such as a doctor makes two or more small holes in a subject, and inserts a cylindrical trocar into each of the holes.

Next, the operator inserts an endoscope, forceps, an electric scalpel or the like into each trocar, and performs surgery while looking at an image captured by the endoscope. Forceps are an example of an instrument for gripping and pulling an internal organ or the like, and remotely controlled. Hereinafter, an instrument, such as an endoscope, forceps and an electric scalpel, for use in endoscopic surgery is referred to as a surgical instrument.

Thus, during endoscopic surgery such as laparoscopic surgery, a surgical instrument such as an endoscope and forceps is inserted into a body of the subject through an incision position, that is, a site where a trocar is to be inserted. Thus, it is necessary for the surgical robot to move the surgical instrument so that a portion of the surgical instrument corresponding to the incision site is immovable.

In view of the above, the present disclosure discloses an example of a surgical robot that allows an operator to confirm whether the surgical instrument is moving so that the portion of the surgical instrument corresponding to the incision site is immovable.

According to some embodiments, a surgical robot for use in endoscopic surgery may comprise at least one of the following components: an arm device that holds a surgical instrument used in endoscopic surgery, a drive device that drives the arm device, a display section for displaying information, and a display processor that displays a relative positional relationship between a site where a trocar is to be inserted and a distal end position of the surgical instrument during surgery on the display section.

This configuration allows an operator such as a doctor to confirm whether the surgical instrument is moving so that a portion of the surgical instrument corresponding to the incision site is immovable. Specifically, the operator such as a doctor may easily and reliably recognize whether the surgical robot recognizes the site where the trocar is to be inserted as an immovable point, in other words, whether surgery by the surgical robot is ready to be performed.

According to some embodiment, a surgical robot may be configured, for example, such that an immovable point setter is provided which recognizes a position of the site where the trocar is to be inserted and stores the recognized position, and the display processor uses the position stored by the immovable point setter as the site where the trocar is to be inserted. This configuration allows an operator such as a doctor to easily and reliably recognize whether the immovable point setter stores the incision position as the immovable point.

According to some embodiment, a drape detector and a second display processor may be provided. The drape detector may detect whether a drape that covers the arm device is attached to the arm device. The second display processor may display a detection result by the drape detector on the display section. This configuration allows the operator to easily and reliably recognize whether surgery by the surgical robot is ready to be performed.

Various embodiments will be described hereinafter.

Arrows indicating directions, hatched lines, etc. shown in the drawings are provided for easy understanding of relationships between the drawings, shapes of members or portions, and others. Accordingly, a configuration of the present disclosure is not limited by the directions shown in the drawings. The drawings with hatched lines do not necessarily show cross-sectional views.

For at least a member or portion described with a reference numeral affixed thereto, there is at least one member or portion unless specified as “one” or the like. In other words, the member or portion may be two or more in number unless specified as “one”. The surgical robot shown in the present disclosure comprises at least components such as members or portions described with reference numerals affixed thereto, and structural portions shown in the drawings.

<1. Configuration of Surgical Robot>

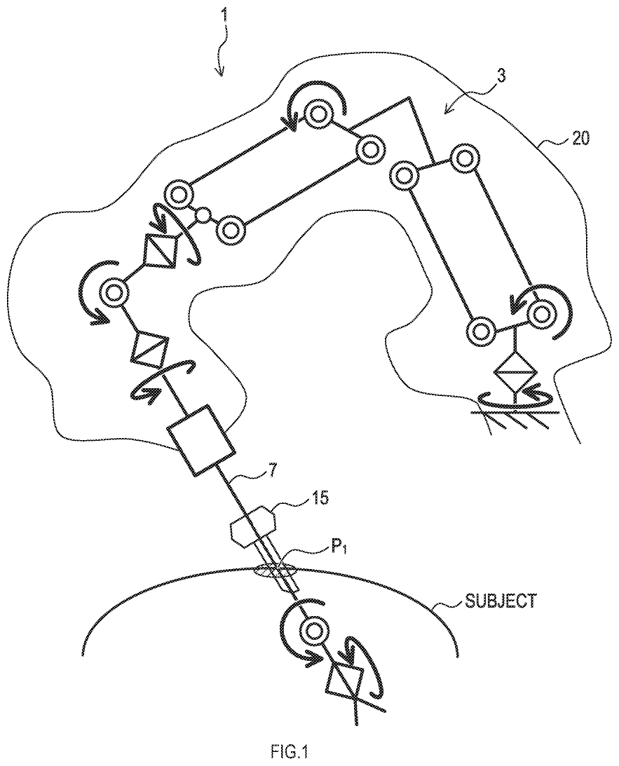

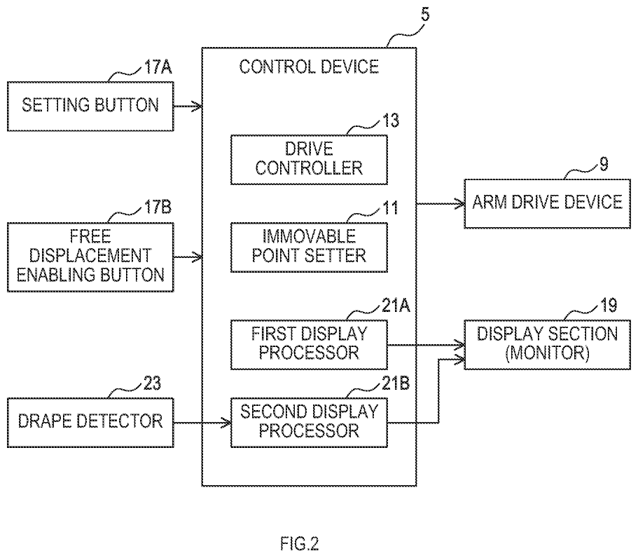

FIG. 1 is an external view of a surgical robot according to some embodiments, and FIG. 2 is a block diagram of the surgical robot according to some embodiments;

A surgical robot for use in endoscopic surgery will be described with reference to FIGS. 1 and 2.

As shown in FIG. 2, a surgical robot 1 may comprise a control device 5, an arm drive device 9, and a display section 19, in addition to a robot arm 3 (see FIG. 1).

<Robot Arm>

The robot arm 3 is an example of an arm device holding a surgical instrument 7, as shown in FIG. 1. Specifically, the robot arm 3 is configured by a link mechanism that has two or more joints and that may change a position of pivot.

The pivot is a position which is an immovable point when the robot arm 3 operates, regardless of a state of the robot arm 3. The surgical instrument 7 is an instrument, such as an endoscope, forceps and an electric scalpel, for use in endoscopic surgery.

The surgical instrument 7 shown in FIG. 1 is a forceps, by way of example. At a distal end of the forceps, a hand part for gripping and pulling an internal organ or the like is provided. The robot arm 3 is covered by a drape 20. The drape 20 may be tubular. The drape 20 is a flexible, non-woven fabric covering member.

<Arm Drive Device (See FIG. 2)>

The arm drive device 9 is an example of a drive device that drives the robot arm 3. The arm drive device 9 according to some embodiments may comprise two or more electric motors, an air pressure cylinder, and a pressure generator.

Each electric motor drives a corresponding joint. The air pressure cylinder applies tension to a wire that drives the surgical instrument 7 (for example, hand part of the forceps). The pressure generator supplies a compressed air to the air pressure cylinder.

<Control Device>

The control device 5 comprises an immovable point setter 11, a drive controller 13, a first display processor 21A, and a second display processor 21B. The control device 5 may be implemented by one or more microprocessors or hardware control logic.

The immovable point setter 11 recognizes a position of a site where a trocar 15 (see FIG. 1) is inserted during surgery (hereinafter, also referred to as an incision position), and stores the recognized position as a pivot P₁.

Hereinafter, a series of operations from recognition of the incision position to storage of the position, etc. by the immovable point setter 11 is referred to as immovable point setting. A state in which the immovable point setting may be performed is referred to as an immovable point setting mode.

The trocar 15 is a cylindrical member to be inserted into a hole incised in a subject. In other words, a surgical instrument 7 such as forceps and an endoscope is inserted into a body of the subject through the trocar 15 inserted to an incision site.

The drive controller 13 uses the position of the pivot P₁ to control operation of the arm drive device 9. Specifically, the drive controller 13 receives a command signal outputted from a master-side input operation device, and activates the arm drive device 9 according to the command signal.

At this time, the drive controller 13 activates the arm drive device 9 so that a portion of the surgical instrument 7 corresponding to the pivot P₁ is immovable. The mater-side input operation device is an example of an input device which is directly operated by an operator such as a doctor.

The first display processor 21A and the second display processor 21B display information on the display section 19. The display section 19 is a monitor that transmits information such as text information and image information to the user. An image captured by the endoscope may be displayed on a monitor separate from the display section 19.

The first display processor 21A displays a relative positional relationship between the incision position, that is, the pivot P₁ and the distal end position of the surgical instrument 7 on the display section 19. According to some embodiments, the first display processor 21A according to some embodiments uses image information such as figures (for example, icons) to display the relative positional relationship on the display section 19. Each icon has a figure which represents the pivot P₁ or the distal end position of the surgical instrument 7.

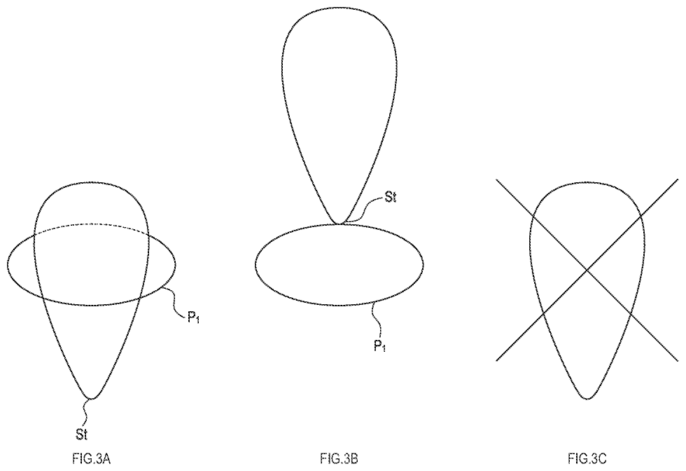

FIGS. 3A to 3C are diagrams showing display examples of the first display processor 21A. Specifically, for example, FIG. 3A shows a case where the distal end position St of the surgical instrument 7 is located inside a body relative to the pivot P₁. FIG. 3B shows a case where the distal end position St of the surgical instrument 7 is located outside the body relative to the pivot P₁. FIG. 3C shows a state in which the immovable point setting is not yet performed.

The second display processor 21B displays a detection result of the drape detector 23 (see FIG. 2) on the display section 19. The drape detector 23 detects whether the drape 20 is attached to the robot arm 3. The drape detector 23 is provided in the robot arm 3.

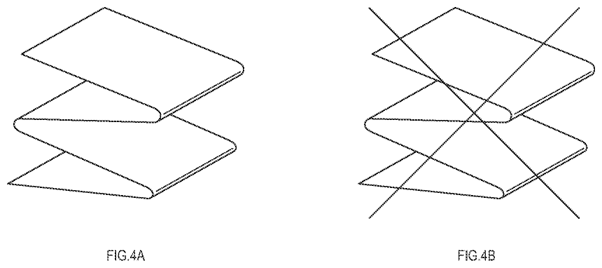

FIGS. 4A and 4B are diagrams showing display examples of the second display processor 21B. If the drape 20 is attached to the robot arm 3, the second display processor 21B displays information (for example, see FIG. 4A) indicating that the drape 20 is attached to the robot arm 3 on the display section 19. If the drape 20 is not attached, the second display processor 21B displays that information (for example, see FIG. 4B) on the display section 19.

<2. Detail of Immovable Point Setter>

The immovable point setter 11 according to some embodiments may execute a position recognition function and a memory function. The immovable point setter 11 uses the position recognition function and the memory function to store the position of the pivot P₁ as an immovable point.

The position recognition function is a function to recognize a distal end position of the surgical instrument 7 held by the robot arm 3. The memory function stores the distal end position recognized by the position recognition function as the pivot P₁. The pivot P₁ stored by the memory function may be, for example, a position recognized by the position recognition function. Also, the position recognized by the position recognition function is not limited to the distal end position of the surgical instrument 7. The position recognized by the position recognition function may be, for example, the incision position which is the position of a site where the trocar 15 is to be inserted during surgery.

The position recognition function according to some embodiments recognizes the distal end position of the surgical instrument 7 by obtaining or calculating a coordinate or the like which indicates the distal end position of the surgical instrument 7 from an attitude of the robot arm 3. The memory function stores the coordinate as the pivot P₁.

To perform the immovable point setting, a surgical instrument equivalent may be used instead of the surgical instrument 7. The surgical instrument equivalent is a member having a shape similar to that of the surgical instrument 7. Specifically, for example, in some embodiments, a rod-shaped or pipe-shaped member may correspond to the surgical instrument equivalent.

The position recognition function and memory function according to some embodiments are implemented by a software, programs that make up the software, and a microcomputer. The microcomputer comprises a CPU, a ROM, a RAM, etc. to run the software. The software is stored in a non-volatile storage section in advance.

The surgical robot 1 has a setting button 17A, a free displacement enabling button 17B and the like, as shown in FIG. 2. The setting button 17A and the free displacement enabling button 17B are provided in at least one of the robot arm 3 and the control device 5. The robot arm 3 corresponds to an example of a slave device, and the control device 5 corresponds to an example of a master-side device.

The setting button 17A is an example of a setting operating section operated by a user. The user is one who performs an immovable point setting work. Specifically, the user is an operator such as a doctor or those who assist surgery. When the setting button 17A is operated, an immovable point setting mode starts or ends.

In other words, if the setting button 17A is operated in a mode other than the immovable point setting mode, the immovable point setting mode is started. If the setting button 17A is operated in the immovable point setting mode, the immovable point setting mode ends.

Specifically, if the setting button 17A is depressed for more than a specified time (for example, three seconds), the immovable point setting mode is started. When the immovable point setting mode is started, the position recognition function is enabled.

When the setting button 17A is depressed less than the specified time (for example, two seconds), the position recognition function is executed and then the memory function is executed. Thereafter, the pivot P₁ is stored as the immovable point, and the immovable point setting mode ends.

The free displacement enabling button 17B is an example of the operating section operated by the user. When the free displacement enabling button 17B is operated, the arm drive device 9 is brought into a free displacement mode. The free displacement mode is a mode in which the robot arm 3 is freely displaceable in accordance with an external force acting on the robot arm 3.

Therefore, in the free displacement mode, the user may freely displace the robot arm 3 by pushing and pulling the robot arm 3. In other words, in the free displacement mode, the user may align the distal end of the surgical instrument 7 with the incision position by pushing and pulling the robot arm 3 without operating the master-side input operation device.

The free displacement mode ends if the free displacement enabling button 17B is operated in the free displacement mode, or when the immovable point setting mode ends. In a state in which the free displacement mode is not started, the robot arm 3 is not displaced even if an external force acts on the robot arm 3.

<Control in Immovable Point Setting Mode>

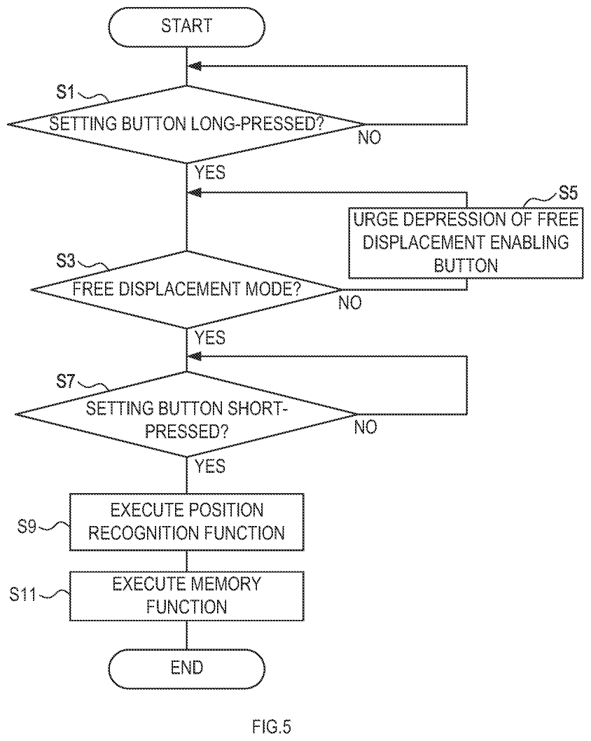

FIG. 5 is a flowchart showing a control of an immovable point setting mode of the surgical robot according to some embodiments. FIG. 5 shows an example control of the control device 5 executed in the immovable point setting mode. The control device 5 determines whether the setting button 17A is depressed for more than a specified time (for example, three seconds) (S1). “(S1)” and the like indicate control step numbers illustrated in FIG. 5.

The control device 5, when determining that the setting button 17A is not depressed for more than the specified time (S1: NO), continues to monitor whether the setting button 17A is depressed, i.e., the process returns to S1. The control device 5, when determining that the setting button 17A is depressed for more than the specified time (S1: YES), determines whether the arm drive device 9 is in the free displacement mode (S3).

The control device 5, when determining that the arm drive device 9 is not in the free displacement mode (S3: NO), urges the user to operate the free displacement enabling button 17B by sound (for example, buzzer) or by a notification device such as a warning light (S5), and the process returns to S3.

The control device 5, when determining that the arm drive device 9 is in the free displacement mode (S3: YES), determines whether the setting button 17A is depressed for less than the specified time (for example, two seconds) (S7).

The control device 5, when determining that the setting button 17A is not depressed for less than the specified time (S7: NO), returns to S7. The control device 5, when determining that the setting button 17A is depressed for less than the specified time (S7: YES), executes the position recognition function (S9) to recognize a distal end position of the surgical instrument 7 held by the robot arm 3, and then executes the memory function (S11) to store the distal end position recognized by the position recognition function as the pivot P₁.

In other words, in some embodiments, when the arm drive device 9 is not in the free displacement mode (S3: NO), the position recognition function and the memory function are virtually disabled.

The control device 5, after storing the pivot P₁ as the immovable point, ends the immovable point setting mode and the free displacement mode, and notifies the user that the pivot P₁ is stored as the immovable point.

<3. Features of Surgical Robot According to Various Embodiments>

In the surgical robot 1 according to some embodiments, the relative positional relationship between the site where the trocar 15 is to be inserted and the distal end position of the surgical instrument 7 during surgery is displayed on the display section 19. This display of the relative positional relationship allows the operator to confirm whether the surgical instrument 7 is moving so that the portion of the surgical instrument 7 corresponding to the incision site is immovable.

In other words, whether the surgical robot 1 has recognized the site where the trocar 15 is to be inserted as the immovable point, that is, whether surgery by the surgical robot 1 is ready to be performed may be easily and reliably recognized by the operator.

The first display processor 21A uses the position stored by the immovable point setter 11 as the site where the trocar 15 is to be inserted. This configuration allows the operator to easily and reliably recognize whether the immovable point setter 11 stores the incision position as the immovable point.

In the surgical robot 1 according to some embodiments, the detection result of the drape detector 23 is displayed on the display section 19. This display allows the operator to easily and reliably recognize whether surgery by the surgical robot 1 is ready to be performed.

The surgical robot 1 according to some embodiments recognizes the position of the site where the trocar 15 is to be inserted during surgery, that is, the incision position, and stores the recognized position as the pivot P₁. Thus, in the surgical robot 1, alignment work between the position of the pivot P₁ and the incision site may be easily performed.

The arm drive device 9 may execute the free displacement mode. Thus, in the surgical robot 1, the user may execute the position recognition function and the memory function after aligning the distal end of the surgical instrument 7 with the incision site. Accordingly, alignment work between the position of the pivot P₁ and the incision site may be easily performed.

### Other Embodiments

The robot arm 3 described with reference to FIGS. 1-5 is configured by a link mechanism that may change the position of pivot. However, embodiments are not limited to the configuration in which the robot arm 3 is configured by a link mechanism that may change the position of pivot. Specifically, for example, in some embodiments, the pivot (hereinafter, also referred to as an immovable point) may be immovable relative to the robot body.

In the description of FIGS. 1-5, the control device 5 comprises the second display processor 21B. However, embodiments are not limited to the configuration in which the control device 5 comprises the second display processor 21B. In some embodiments, the second display processor 21B may be provided in a component other than the control device 5, or the second display processor 21B may be omitted, etc.

In the description of FIGS. 1-5, if the arm drive device 9 is not in the free displacement mode (S7: NO), the control device 5 disables the position recognition function and the memory function. However, embodiments are not limited to the configuration in which the control device 5 disables the recognition function and the memory function if the arm drive device 9 is not in the free displacement mode (S7: NO).

Specifically, for example, in some embodiments, even in a mode other than the free displacement mode, the control device 5 may enable the position recognition function and the memory function. In this case, the control device 5 may use the master-side input operation device to align the distal end of the surgical instrument 7 with the incision position.

The immovable point setter 11 according to FIGS. 1-5 obtains the coordinate representing the distal end position of the surgical instrument 7 from the attitude of the robot arm 3 to recognize the distal end position. However, embodiments are not limited to the configuration in which the coordinate representing the distal end position of the surgical instrument 7 is obtained from the attitude of the robot arm 3 to recognize the distal end position. Specifically, for example, in some embodiments, the distal end position may be recognized with an image analysis technique that uses a 3D camera such as a stereo camera and a depth camera to determine the distal end position.

In the description of FIGS. 1-5, the user recognizes the distal end of the surgical instrument 7 or a surgical instrument equivalent, in a state in which the distal end is aligned with the incision position, to recognize the incision position. However, embodiments are not limited to the configuration in which the user recognizes the distal end of the surgical instrument 7 or a surgical instrument equivalent, in a state in which the distal end is aligned with the incision position. Specifically, for example, in some embodiments, a laser light may be applied to the incision position, and the applied position may be recognized by an image analysis technique.

In the description of FIGS. 1-5, when the free displacement enabling button 17B is operated, the free displacement mode is started. However, embodiments are not limited to the configuration in which, when the free displacement enabling button 17B is operated, the free displacement mode is started. Specifically, for example, in some embodiments, at the same time as the immovable point setting mode is started, the free displacement mode may be automatically started.

Various embodiments have been described above with reference to the drawings. However, it is to be understood that the present disclosure is not limited to the above embodiments, but various changes and modifications may be made therein without departing from the spirit and scope thereof as set forth in appended claims.

What is claimed is:

1. A surgical robot comprising:
an arm device that holds a surgical instrument used in endoscopic surgery;
a drive device that drives the arm device;
a display section; and
a first display processor that displays, on the display section, a relative positional relationship between a site where a trocar is to be inserted and a distal end position of the surgical instrument.

2. The surgical robot according to claim 1, further comprising:
a control device configured to implement an immovable point setter that recognizes a position of the site where the trocar is to be inserted and stores the position,
wherein the first display processor uses the position that is stored as the site where the trocar is to be inserted.

3. The surgical robot according to claim 2, further comprising:
a drape detector that detects whether a drape that covers the arm device is attached to the arm device; and
a second display processor that displays a detection result of the drape detector on the display section.

4. The surgical robot according to claim 1, further comprising:
a drape detector that detects whether a drape that covers the arm device is attached to the arm device; and
a second display processor that displays a detection result of the drape detector on the display section.

5. The surgical robot according to claim 1, wherein the arm device comprises two or more joints that are movable to change a position of a pivot.

6. The surgical robot according to claim 1, wherein a position of the site where the trocar is to be inserted is recognized and stored in an immovable point setting mode of the surgical robot.

7. A surgical robot comprising:
a robot arm that holds a surgical instrument used in endoscopic surgery;
a drive device that drives the robot arm;
a display; and
a processor or hardware logic that displays, on the display, a relative positional relationship between a trocar site and a distal end position of the surgical instrument.

8. The surgical robot according to claim 7, wherein the processor or hardware logic recognizes a position of a site where a trocar is to be inserted and stores the position,
wherein the trocar site comprises the position.

9. The surgical robot according to claim 8, further comprising:
a drape detector that detects whether a drape that covers the robot arm is attached to the robot arm,
wherein the processor or hardware logic displays, on the display, a detection result of the drape detector.

10. The surgical robot according to claim 7, further comprising:
a drape detector that detects whether a drape that covers the robot arm is attached to the robot arm,
wherein the processor or hardware logic displays, on the display, a detection result of the drape detector.

11. The surgical robot according to claim 7, wherein the robot arm comprises two or more joints that are movable to change a position of a pivot.

12. The surgical robot according to claim 7, wherein a position of the trocar site where a trocar is to be inserted is recognized and stored in an immovable point setting mode of the surgical robot.

13. A surgical robot comprising:
a robot arm that holds a surgical instrument used in endoscopic surgery;
a drive device that drives the robot arm;
a display; and
a processor or hardware logic that:
based on a setting button being depressed, starts an immovable point setting mode that allows free displacement of the robot arm;
based on the setting button being depressed again, recognizes a position of a site where a trocar is to be inserted, stores the position as a trocar site, and ends the immovable point setting mode; and
displays, on the display, a relative positional relationship between the trocar site and a distal end position of the surgical instrument.

14. The surgical robot according to claim 13, further comprising:
a drape detector that detects whether a drape that covers the robot arm is attached to the robot arm,
wherein the processor or hardware logic displays a detection result of the drape detector on the display.
